(19) United States
(12) Patent Application Publication
(10) Pub. No.: US 2009/0209846 A1
(43) Pub. Date: Aug. 20, 2009

(54) Apparatus and method for real-time motion-compensated magnetic resonance imaging

(76) Inventor: Roland Bammer, Palo Alto, CA (US)

Correspondence Address: LUMEN PATENT FIRM, 2345 YALE STREET, SECOND FLOOR, PALO ALTO, CA, 94306, US

(21) Appl. No.: 11/823,469

(22) Filed: Jun. 26, 2007

Related U.S. Application Data
(60) Provisional application No. 60/817,490, filed on Jun. 28, 2006.

(51) Int. Cl. A61B 5/055; G06K 9/00

(52) U.S. Cl. 600/421; 382/131

(57) ABSTRACT

The present invention provides an apparatus and method for real-time motion compensated magnetic resonance imaging (MRI) of a human or animal. The apparatus includes one or more magnetic-resonance compatible cameras mounted on a coil of the MRI device, a calculation and storage device, and an interface operably connected to the MRI device and the calculation and storage device. The apparatus may also include a set of magnetic resonance compatible markers, where the markers are positioned on the human or animal. Alternatively, the apparatus may use a facial recognition algorithm to identify features of the human or animal. For the present invention, the frame of reference is defined by the animal or human being imaged, instead of the typical magnetic resonance coordinate system. Based on continuous positional information, the apparatus controls the magnetic resonance scanner so that it follows the human or animal's motion.

### CROSS-REFERENCE TO RELATED APPLICATIONS

This application claims priority from U.S. Provisional Patent Application No. 60/817,490, filed Jun. 28, 2006, which is incorporated herein by reference.

### STATEMENT REGARDING FEDERALLY SPONSORED RESEARCH OR DEVELOPMENT

This invention was made in part with government support under grant nos. R01-EB002771, R21 EB 00680 and P41RR09784 awarded by the National Institutes of Health (NIH). The government has certain rights in this invention.

### FIELD OF THE INVENTION

The present invention relates generally to medical imaging. More particularly, the present invention relates to an apparatus and method for real-time motion-compensated magnetic resonance imaging.

### BACKGROUND

Due to the sequential nature of the magnetic resonance imaging (MRI) acquisition process, motion artifacts remain one of the most prevalent confounders of MR studies. Motion artifacts cause repeated or non-diagnostic exams, contributing to increased exam costs and diminishing clinical yield. The ability to determine and correct for motion becomes paramount in order to maintain MRI's excellent diagnostic quality.

Thus far, much research in MRI has focused on motion compensation, usually through specialized k-space trajectories and/or additional MR data collection. However, these methods must still be developed on a per-sequence basis; no general correction scheme yet exists. Further, most methods are retrospective, and cannot compensate for spin history effects; only some can synthesize the missing k-space data after correction.

Other methods have used IR cameras positioned outside the bore of the MRI device along with markers attached to the patient. As these cameras were not MR-compatible, they needed to be positioned as far away from the MRI device as possible. This setup has pronounced visibility concerns, as the cameras must view any markers through the length of the bore, and past the patient and any coils. These are extreme limitations, especially for patients with large girths and long bore magnets. Accordingly, there is a need in the art to develop prospective methods of motion compensating magnetic resonance imaging.

### SUMMARY OF THE INVENTION

The present invention provides a method and apparatus for real-time motion compensation of MRI. For this invention, the frame of reference is defined by the human or animal being imaged, instead of the typical MR-based coordinate system. Based on continuous positional information the apparatus controls the MR scanner so that it follows the human or animal's motion.

The apparatus according to the present invention contains several components. A first component includes one or more MR-compatible cameras, which mounts on an MR coil of an MRI device. Preferably, the cameras are digital, where both the cameras and associated control electronics are MR-compatible and can be mounted on standard radio frequency (RF) MR coils. Also preferably, the cameras do not interfere with the polarization field (B0 field) or the transmit/receive field (B1 field). In addition, the cameras and associated electronics can preferably provide positional information at least every 20-30 ms. A second component is a calculation and storage device. The calculation and storage device retrieves images from the cameras, identifies markers positioned on the human or animal or features of the human or animal, determines the position of the markers or features in 3 dimensions, and determines the rotation and translation of the markers or features. The calculation and storage device may be, e.g., a computer, software, MRI workstation, etc. A third component is an interface, which is operably connected to both the calculation and storage device and the MRI device. This interface preferably controls the geometrical prescriptions of the MRI device, i.e. slice orientation and position, in real-time by altering the rotation processor and transmit/receive frequency/phase. The apparatus according to the present invention may also include a calculation device that automatically modifies the geometrical prescriptions of the MRI device to realign scans after a subject has left and re-entered the scanner, as well as a visual patient monitor and patient motion curves display for patient surveillance.

The apparatus may also include a set of MR compatible markers, where the markers are positioned on the human or animal. Preferably, the markers are small (less than about 5 mm) and passive, i.e. they require no wires or connections. The markers may also be of varying diameter. These markers may be imaged both by the MR scanner as well as the cameras in order to calibrate the two coordinate systems. One form of MR visibility is a hollow marker filled with an MR-visible substance.

In another embodiment, a single camera is used, instead of a multiplicity of cameras. In this embodiment, the tracking marker contains a number of known dimensions, from which depth information may be calculated from a single observation of the object. The remainder of the apparatus remains unchanged in this embodiment.

In another embodiment, the apparatus does not include markers. In this embodiment, the calculation and storage device identifies features of the human or animal, determines the position of the features of the human or animal in three dimensions, and determines the rotation and translation of the features. Features of the animal or human are identified using a facial recognition algorithm. The position of these features is then determined, and the rotation and translation of the features is then determined.

The present invention also provides a method of motion compensating MRI of a human or animal in real time. In one embodiment, at least one MR-compatible camera is mounted on an MR coil of an MRI device. Next, a set of MR-compatible markers are positioned on the human or animal. The cameras then acquire an image of the markers, the markers are identified, and the position of the markers in three dimensions is determined. Next, the rotation and translation of the markers is determined and the geometric prescriptions of the MRI device are modified based on the determined rotation and translation. Preferably, this method includes a parallel imaging reconstruction method that functions with data acquired during prospective motion correction when the coil sensitivity varies in space (based on the patient defined frame of reference). Also preferably, this method includes imaging the markers using MR imaging, and transforming the positions of the markers from a coordinate system of one of the cameras to a coordinate system of the MRI device.

In another embodiment, at least two MR-compatible cameras are mounted on an MR coil of an MRI device. Next, the cameras acquire an image of the head of the human or animal, features of the head are identified using a facial recognition algorithm, and the position of the features in three dimensions is determined. Next, the rotation and translation of the features is determined and the geometric prescriptions of the MRI device are modified based on the determined rotation and translation. Preferably, the identifying is based on at least one of Haar classifiers or a CAMSHIFT algorithm. Also preferably, this method includes a parallel imaging reconstruction method that functions with data acquired during prospective motion correction when the coil sensitivity varies in space (based on the patient defined frame of reference).

### BRIEF DESCRIPTION OF THE FIGURES

The present invention together with its objectives and advantages will be understood by reading the following description in conjunction with the drawings, in which:

FIG. 1 shows an example of an apparatus according to the present invention.

FIG. 2 shows an example of a circuit diagram for a camera system according to the present invention.

FIG. 3 shows examples of a top view (A) and a side view (B) of a first setup for a set of markers and a side view (C) of a second setup for a set of markers according to the present invention.

FIG. 4 shows an example of an MR-compatible CCD imager according to the present invention.

### DETAILED DESCRIPTION OF THE INVENTION

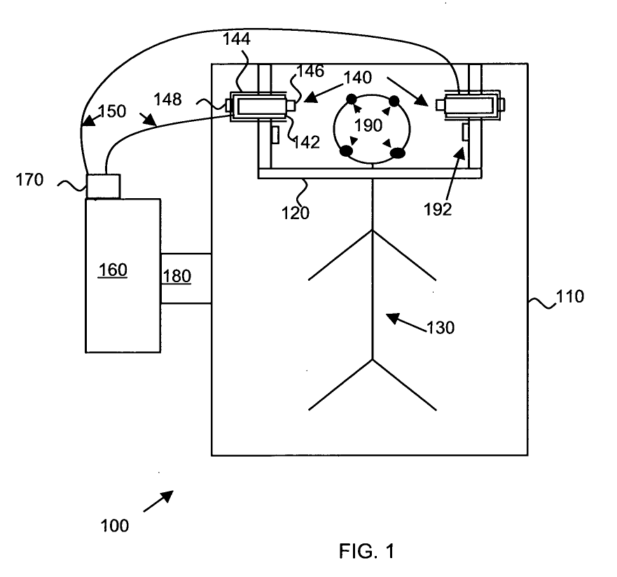

FIG. 1 shows an example of an apparatus according to the present invention. Apparatus 100 includes magnetic resonance imager 110, having coil 120, in which human or animal 130 is placed. Preferably, coil 120 is a head coil. At least one magnetic-resonance compatible camera 140 is mounted on coil 120. Each camera 140 further preferably includes a protective housing 142. Protective housing 142 is preferably covered by grounded copper shielding 144, such that a lens 146 of each camera 140 is uncovered by shielding 144. Alternatively, lens 146 may be covered by a visibly transparent but RF-shielded copper mesh. Preferably, housing 142 further includes thermocouples, where the thermocouples 148 are attached to housing 142. Apparatus 100 also preferably includes a control and transfer system 150. System 150 transfers data from cameras 140 to a calculation and storage device 160. Preferably, apparatus 100 also includes a processor 170, which provides a line conditioning and voltage protection circuit for calculation and storage device 160. Finally, apparatus 100 includes an interface 180, which is operably connected to calculation and storage device 160 and magnetic resonance imager 110.

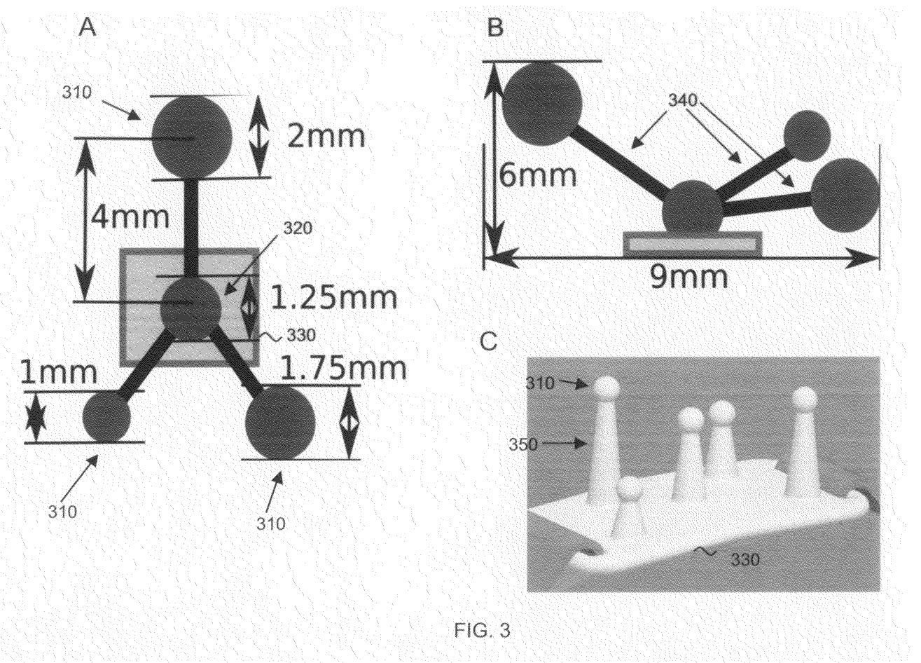

In one embodiment, apparatus 100 is used without a marker system. In this case, calculation and storage device 160 identifies features of human or animal 130, determines the position of these features in three dimensions, and determines the rotation and translation of these features. In another embodiment, a set of markers 190 is used, as shown in FIG. 3. Preferably, markers 190 are of varying diameter, as shown in FIG. 3. In this embodiment, apparatus 100 preferably includes at least one light source 192 for illuminating markers 190. Preferably, apparatus 100 contains one light source in proximity to each camera 140, as shown in FIG. 3. The light source and camera system can be sensitive to the whole spectrum of light or just to a certain bandwidth (e.g. infrared).

### MR-Compatible Camera System

In order to determine 3D position, at least two cameras must simultaneously view an object. The stereo camera system preferably mounts to a central vertical rung on an RF MR coil, such that each camera will view through gaps between the rungs. Preferably, the stereo camera system is designed to mount to a 28-30 cm head coil for adults or coils of smaller diameter for children. As 3D position may be determined anywhere both cameras can image the markers, position may be tracked over at least a 90° roll. In one embodiment, a center mounted polycarbonate or PMMA rig with cameras mounted 15 cm apart, 30° off horizontal, gives good coverage of the patient's head, even under severe rotations and translations. The rig is preferably precision milled to ensure a snug fit as well as accurate, precise, and stable mounting of the cameras on the coil. Crosshairs may be placed on the top and sides of the rig to assure true axial alignment and repeatable positioning of the camera on the coil. In addition, vibrations induced from switching gradient coils may be damped by foam material placed on the coil rungs. Cameras are preferably set back slightly, to maximize field of view (FOV) at this close range. Wide-angle, 90° lenses are preferably used as well, for the same reason. This setup maintains compatibility with the standard GE quadrature head coil, MEDRAD 8-channel neurovascular array, and the MRI devices 8-channel head coil, requiring changes only in the mount. Each camera has an unoccluded view of the patient's face with rotations up to ±45° left and right, and translations up to those that impact the coil itself. Each camera is preferably a low-cost, low-voltage CCD imager. Modern CCD cameras and lenses have excellent signal to noise ratio (SNR) and light sensitivity, with low image distortion.

The housing for each camera in the system is preferably shrouded in grounded copper shielding, leaving only the lens uncovered, to avoid MR signal perturbation by the operation of the camera's electronics, as well as to protect the camera signal during RF transmission. Alternatively, the lens may be covered by a visibly transparent but RF-shielded copper mesh. As CCD imagers are sensitive to both visible and IR light an IR imager may also be used by placing an IR filter in front of the lens. When in IR mode, IR LEDs around each camera may invisibly illuminate the scene, increasing both SNR and contrast.

Preferably, data is transferred from the cameras via video using either an analog or a digital control and transfer system. For ease of integration and signal compatibility with MR, each camera preferably uses USB compatible signaling, although other signaling methods known in the art are possible. USB uses a pair of differential signaling wires, with signal differences of up to 3.6 V in full speed mode. The 12 Mbit rate changes state every 83 ns. The clock rate will not interfere with the center-frequency of high-field MRI. Further, a rapidly switching gradient, for example with low resolution Echo-planar imaging (EPI), switches about 7,500 times slower, every 0.6 ms. Full speed USB has enough bandwidth to transfer Quarter Common Intermediate Format (QCIF) frames at 60 frames per second, giving a time resolution of 16.7 ms. USB, unlike IEEE-1394, does not support native device synchronization. Therefore, each camera is preferably placed on a separate USB controller, with software synchronization performed by a calculation device. In an alternative embodiment, IEEE-1394 communication may be used. IEEE-1394 uses two differential signal pairs, and may run at 100, 200 or 400 MHz. One may be selected to avoid main carrier frequency crosstalk at any field strength. IEEE-1394's increased bandwidth allows higher resolution imaging while maintaining a high frame rate. In another embodiment, analog video signaling using either the NTSC or PAL video formats may be used by the camera(s). The calculation device then uses a frame grabbing device in order to digitize each image.

Cabling is preferably made using three sets of copper core aluminum shielded twisted pair wiring. Shielded twisted pair offers great magnetic coupling rejection and low loss up through several MHz. In an alternate embodiment, coaxial or triaxial cables are used, which could further be RF choked by cutting the outer shield bridging it to the inner shield at λ/4 intervals.

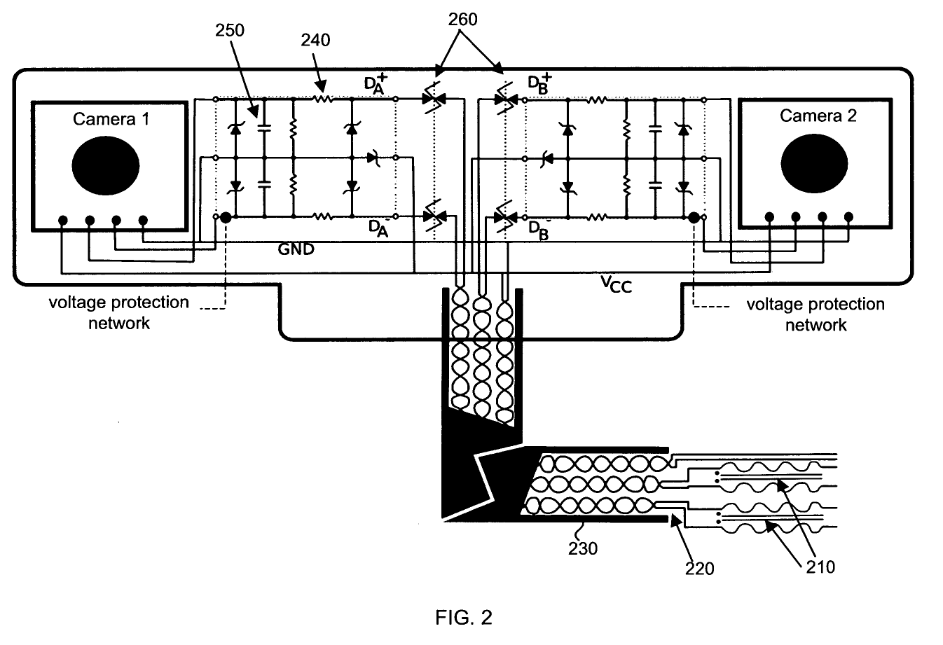

Although the differential signaling on the twisted-pair wires will reject most coupling from the switching of the B1 field, additional circuit protection is still warranted to avoid damage to either the camera's or a data reception port from induced transient signals. FIG. 2 shows a simplified line conditioning and voltage protection circuit diagram for a stereo camera system according to the present invention. Passive common-mode chokes 210 are preferably installed between the differential signaling pairs 220 of the communication cabling 230 in order to reject common mode noise, without perturbing the differential signal. Due to the choke's inductance, it is preferably used on the near end of the line, rather than in the magnet's bore. The relatively low clock rate of full speed USB allows the use of inline ceramic resistors 240 and aluminum capacitors 250 for electromagnetic interference (EMI) noise filtering, electrostatic discharge (ESD) protection, and line termination, and is available in a small IC package from a number of manufacturers. Further over-voltage protection may be provided by low voltage polymer dual-rail clamps 260, an ultra low capacitance version of the clamping diode. Finally, should spurious noise spikes enter the line, both protocols can handle single and double bit errors via cyclical redundancy checks (CRCs), and can re-request corrupted or lost packets.

An alternate embodiment uses optical transmission lines. In this embodiment, an optocoupler converts the electronic video signal into optical signals as close to the camera as possible in order to avoid interferences between the video feed and the MR scanner. Another optocoupler exists at the far end of the optical fiber line, which converts the optical signaling back to electrical signal for the calculation device.

Successful MR image formation relies on homogenous B₀ and B₁ fields over the volume of interest. Any magnetically susceptible object in these fields will perturb them. Thus, the stereo camera setup is preferably constructed of non-susceptible materials, e.g. most plastics, and conducting components are preferably made of less susceptible metals, such as copper, brass, and aluminum. Further, components are kept as far away from the imaging field as possible. Higher-order B₀ shimming can partially alleviate B₀ inhomogeneity from both patient and camera. In case of residual B₀ perturbations, a small shim coil set preferably surrounds each camera, in order to correct local field inhomogeneities.

The rapidly switching gradient fields will induce currents in conducting components, increasing their temperature. Component failure through heating is an unlikely event, as the least tolerant device, the CCD, is rated for operation through 85° C. However, components might become warm to the touch, so they will remain shrouded inside the camera system to avoid possibility of burns to a user or patient. A number of thermal issues related to performance may also occur. As they heat, components inside the camera emit more infrared light. While visible-light images can be IR filtered to remove these wavelengths, decreased SNR in the IR images will be unavoidable. Increased component temperatures might also warp the stereo setup, invalidating the extrinsic calibration. Therefore, the stereo camera system preferably includes thermocouples mounted to the shielding, to monitor temperature during operation in a wide variety of scan protocols. This will ensure the system does not go above safe operating temperatures.

### Marker Design and Tracking

Before calculating real-world locations of markers, the markers must be located in each image frame. An IR based marker design, where an array of small IR reflective spots is placed on the forehead of the subject greatly simplifies this task. In this case, the scene is preferably illuminated by a small array of 6 (3 per camera) GaAlAs IR LEDs near the camera, resulting in an image with great contrast between the markers and the background. Each LED preferably draws a maximum of 75 mW at 1.5V and operates at up to 85° C. The total power drawn by three LEDs is preferably less than one USB low-power device, and thus may be drawn from the USB V_{cc} power line.

The array of markers is preferably a non-metallic, completely passive device (although an active light emitting source should not be excluded), requiring no wires or connections. Patient safety and image quality will thus not be compromised. The reflective array is preferably a known 3D pattern of several reflective spheres of varying radii. Two examples are shown in FIG. 3. FIGS. 3A and B show an embodiment where spheres 310 of varying but known sizes and spacings are attached to a central sphere 320, which in turn may be attached through mount 330 to a patient's forehead. Preferably, each sphere can be used to determine position changes. The variation in size allows a computer algorithm to more easily determine the orientation of the array, and the use of varying heights and lengths of interconnecting segments 340 reduces occlusions from rotations. FIG. 3C shows a model of marker array set as it would be produced from a photolithographer. In this case, spheres 310 are attached to mount 330 through cone-shaped segments 350. Note that the radii and distances shown in FIG. 3 are for illustrative purposes only, and are not meant to be limiting. At least four spheres are preferably used so that 3D motion may be calculated even with a single occlusion. Though the array is typically less than 1 cm in each dimension, the spheres preferably cover several pixels for easier object detection, but remain small when imaged to aid estimation of small motions. The array's positioning on the patient is irrelevant, as long as it has a fixed relation to the patient, and the stereo camera system remains fixed to the coil. This setup is beneficial, as the patient may be unloaded for medical interventions, and then reloaded into the magnet. As long as the markers remain affixed, the system may continue the examination, updating the MR coordinate system as necessary, but without need for another landmark and scout acquisition.

In another embodiment, the spheres in the marker array are hollow, and are filled with, e.g., Gd-doped water, so that they may also be imaged using the MR scanner. Concurrent imaging of the markers by the camera and the MR scanner can be used to rectify the differences between the camera coordinate system and the MR scanner coordinate system. One can also use substances that are invisible for conventional MR, but visible to special MR parameter setting or pulse sequences (e.g. ultrasort TE MRI) to avoid seeing marker on regular MR scans.

The initially determined position is the reference point that determines the original MR frame of reference. Later position changes from this original position require the update of the MR reference system. The location of the markers in an image may be determined, e.g., using a mean shift algorithm with a cascade of boosted Haar classifiers, which is a robust gradient climbing scheme that finds the peak of the probability distribution function of the image histogram. After each reflective sphere has been located in the image from each camera, the 3D location of each may be calculated using epipolar geometry using techniques known in the art.

The placement of an IR reflective array on the subject does add complexity to use of the system. Therefore, in an alternative embodiment, automatic face identification and fiducial extraction may be used. Face detection via Haar classifiers is a mature technology, which has found numerous applications, such as scanning crowds to recognize known criminals. Face detection may be combined with a CAMSHIFT algorithm, an efficient algorithm that uses continually adaptive probability distributions, and was designed for facial tracking.

### Modifying the Geometrical Prescriptions of the MRI Device

Current clinical MR scanners contain advanced gradient and RF control systems. These systems already allow the adjustment of sequence parameters during acquisition in near real time. In this invention, the tracked position is used to modulate the MR scan acquisition.

The output from the above algorithms is 3D locations in real-world coordinates. However, unlike normal MRI in which the FOV is determined relative to the physical gradient system, this invention uses the patient to determine the frame of reference. In other words, the FOV is “locked” to the patient, e.g. to the patient's head. When motion of the head occurs, the scanner FOV must be updated. Changes in the coordinates of the head location take place among 6 degrees of freedom, i.e. rotation and translation in each of 3 dimensions. These must be translated into changes in the MR acquisition system.

In order to more easily prescribe and use oblique imaging axes, scanners use a global set of rotation matrices, which operate on all three gradient axes directly. These rotation matrices are applied as the final step before the gradient amplifiers generate the requested currents. A pulse sequence plays out particular gradient strengths for the logical gradients Gₓ, G_{y}, and G_{z} which then get routed through a 3D rotation matrix R, g′=Rg, and the result is sent to the gradient amplifiers that provide the current to energize the physical gradient coils. Therefore, rotations in the patient's head may be compensated by updating these rotation matrices as necessary.

Translations must be handled differently based on the dimension. A translation along the slice-encoding direction requires a simple change in the frequency of the RF excitation. For a translation in this slice-encoding direction of Δz, the frequency is changed by (γ/2π)·ΔzG_{z}, where γ is the Larmor frequency. Translations in the frequency encoding direction require modulating the frequency of the data acquisition board by (γ/2π)·ΔxG_{X}, where Δx is the translation. Translations in phase encoding direction could be handled by adding a phase ramp in this direction to the data upon reconstruction. However, to keep motion compensation confined to the scanner hardware, for a shift of Δy the phase offset in the receiver board can simply be offset by 2πΔy/FOV_{y} per each phase encoding step, where FOV_{y} is the phase encoding direction's field of view. Each of these frequency and phase offsets, once calculated, may be updated by accessing a scanner control bus.

If communication of k-space locations is established between the pulse sequence and the reference frame update, an alternate translation correction is possible using the data acquisition board, even in non-Cartesian acquisitions. As data arrives, it may be demodulated using the following formula:

$${m}_{corr} ( t ) = m ( t ) {e}^{j 2 π ( \frac{{k}_{x} ( t ) \cdot δ x}{( {k}_{x , max} - {k}_{x , min} ) \cdot {FOV}_{x}} + \frac{{k}_{y} ( t ) \cdot δ y}{( {k}_{y , max} - {k}_{y , min} ) \cdot {FOV}_{y}} + \frac{{k}_{z} ( t ) \cdot δ z}{( {k}_{z , max} - {k}_{z , min} ) \cdot {FOV}_{z}} )} . \qquad [ 1 ]$$

### Interface

An interface retrieves updated position information from the (stereo) camera system and uses this information to modify the scanner's parameters. In order to properly calculate frequency and phase offsets from position information, as well as to synchronize updates with acquisitions, two-way communication is needed. The interface must acquire from the control software values such as the imaging field of view, in-plane resolution, slice thickness, Larmor frequency, gradient strengths, and initial offsets from isocenter, as well as timing information, such as the TR. This information may be used to calculate updated scan parameters, which will be sent to the scanner's hardware.

In one embodiment, this interface is a software process that is built inside a pulse sequence's specific software program and runs on the host computer. As this process is part of a specific sequence's software, it allows simple communication of the aforementioned sequence values to the interface. This further allows the use of manufacturer provided software functions for setting parameters such as transmit and receive frequencies and rotation matrices on the spectrometer unit.

The lag time for applying and responding to software-based update commands to the system rotation matrix, receiver, and exciter is negligible, at 4 μs. Synchronization with the sequence will also be aided, as the looping constructs in the pulse sequence may be modified at will. These constructs start, for example, immediately prior to excitation and each reception task, and would be an ideal location at which to update the current patient position.

The timing of some sequences, such as steady-state free precession (SSFP) or spoiled gradient-echo (SPGR), requires extremely rapid sequence updates, on the order of every several milliseconds. In these cases, the position information will not be available as often as these updates. The position retrieval will handle this discrepancy by simply always fetching the latest calculated position. This position may be as much as 25 ms old; however, the difference in the position possible in this interval is still quite small.

In another embodiment, the interface is a hardware interface that can access and set the above-described values on the scanner's hardware interface. This embodiment avoids the need to edit each sequence to add motion correction and increases compatibility with other installation sites. This hardware interface may join an Ethernet bridging the scanner console, where the pulse sequence starts, with a VersaModule Eurocard (VME) bus. The tracking processor may construct transmission control protocol (TCP) packets, sending them to a BIT3 interface, which will deliver the packet's payload to the VME bus. The requisite updates may all be handled via VME bus packets. Rotation matrices may be updated by constructing the requisite bit packet, which is placed on the communication bus. Frequency and phase information may be updated via VME bus-addressable registers, which are latched to an accumulator. This setup allows changes to the values to be set, rather than needing the absolute frequency or phase. That is, frequency offset can be calculated in Hz, and the final frequency is shifted by this amount, removing the need to use values such as center frequency or sampling bandwidth in the calculations.

To ensure coordinate system updates occur only during quiescent periods, the tracking computer preferably monitors the VME bus, and will only update between the end-of-sequence (EOS) packet, and the start-of-sequence interrupt (SSI). The EOS packet occurs when a pulse sequence informs the sequencers that it has finished modulating its waveforms and settings. The SSI packet begins the actual playout of the newly-formed sequence. Each sequence is usually the waveforms to perform one acquisition, and the SSI packet occurs once per slice, per repetition time (TR). Thus, a relatively long quiescent period usually exists between them, when scan updates may occur. Even in very short TR sequences, as in SSFP or SPGR, a minimum of approximately 600 μs exists between these packets.

### Parallel Image Reconstruction Method

In the presence of motion, the stationary radio-frequency receive coil array enters the reconstruction problem as a positional variation. In other words, during the course of data acquisition the coil sensitivity information varies dynamically and, depending on the extent of motion, this fluctuation can impair the result of parallel imaging reconstructions. Thus, in a preferred embodiment, a parallel imaging reconstruction algorithm is used along with the prospective motion correction methods described above. This algorithm may be, for example, GRAPPA, SENSE, mSENSE or an augmented generalized SENSE (GSENSE), an iterative reconstruction that allows for parallel imaging reconstruction of arbitrary sampled k-space data. Augmented GSENSE uses the patient as the frame of reference and can correct the differing coil sensitivities seen by each acquisition. This is done by corresponding rotations and translations of the coil sensitivity, S_{c}→(rot,trans)_{l→s}_{l,c} in the encoding matrix, E, with E_{(l,q,c),(p)}=s_{c}(rₚ)·exp(k_{l,q}), so that the final image can be computed by solving (E^{H}E)v⁽ⁿ⁾=E^{H}k using the conjugate gradient algorithm. Here, l is the index for each profile/interleaf, c is the coil index (1 . . . nc), p is the position index (1 . . . N×N), and q is the k-space data sample index (1 . . . k).

### Example

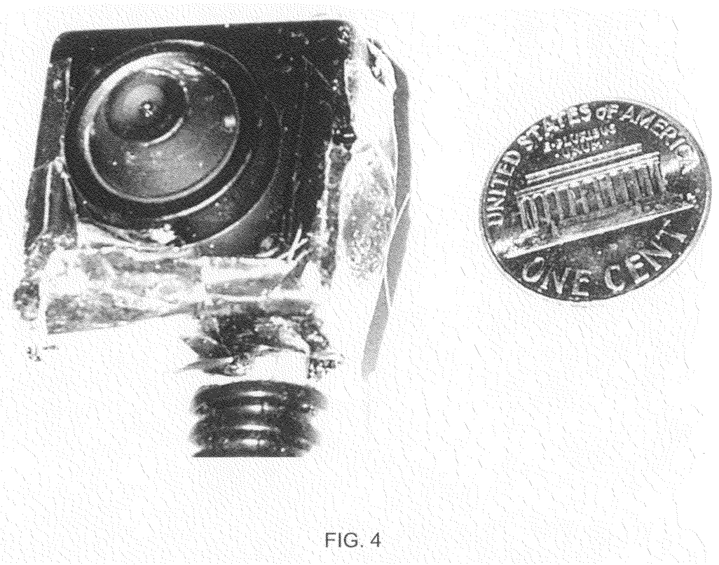

FIG. 4 shows an example of an MR-compatible CCD imager according to the present invention. The camera shown in FIG. 4 was constructed from a pinhole lens, wide angle (˜75°) “spy cam” with automatically adjustable iris for variable lighting conditions. The camera was selected amongst several other types because of its high image quality, lack of additionally required lighting (i.e. the illumination inside the bore is sufficient), and the negligible magnetic susceptibility changes after highly magnetically susceptible parts were removed, and the remaining circuitry was shielded. Technical specifications of the camera were as follows: ⅓″ solid state interline non-CMOS CCD-BW chip; scanning system: EIA 525 lines, 2:1 interlacing; shutter/exposure: automatically selected, 1/60-1/100,000 sec; luminance SNR: >45 db; Sensitivity: 0.1 Lux; input voltage: 9-12 volts @ 100 mA; size: 25×25 mm²; 380 TV lines; 3.6 mm pinhole lens. This camera currently operates inside the bore of a 1.5 T MR magnet.

As one of ordinary skill in the art will appreciate, various changes, substitutions, and alterations could be made or otherwise implemented without departing from the principles of the present invention. For example, the method of the present invention could be used by an anesthesiologist, such that sedation can be re-administered as needed. Accordingly, the scope of the invention should be determined by the following claims and their legal equivalents.

What is claimed is:

1. An apparatus for real-time motion compensated magnetic resonance imaging of a human or animal, comprising:
a) one or more magnetic-resonance compatible cameras, wherein said cameras are mounted on a magnetic resonance coil of a magnetic resonance imaging device;
b) a calculation and storage device; and
c) an interface, wherein said interface is operably connected to said calculation and storage device and said magnetic resonance imaging device.

2. The apparatus as set forth in claim 1, further comprising a set of magnetic resonance compatible markers, wherein said markers are positioned on said human or animal.

3. The apparatus as set forth in claim 2, further comprising a magnetic-resonance compatible light source for illuminating said markers, wherein said light source is situated in proximity to said one or more magnetic-resonance compatible cameras.

4. The apparatus as set forth in claim 2, wherein said markers have varying diameters.

5. The apparatus as forth in claim 2, wherein said calculation and storage device comprises an algorithm for identifying said markers, an algorithm for determining the position of said markers in three dimensions, and an algorithm for determining the rotation and translation of said markers.

6. The apparatus as set forth in claim 1, further comprising a housing for each of said one or more magnetic-resonance compatible cameras.

7. The apparatus as set forth in claim 6, wherein said housing is covered by grounded copper shielding, and wherein a lens of each of said one or more cameras is uncovered by said grounded copper shielding or covered by a visibly transparent but RF-shielded copper mesh.

8. The apparatus as set forth in claim 6, wherein said housing further comprises thermocouples, wherein said thermocouples are mounted to said housing.

9. The apparatus as set forth in claim 1, further comprising a control and transfer system, wherein said control and transfer system transfers data from said cameras to said calculation and storage device.

10. The apparatus as set forth in claim 9, further comprising a processor, wherein said processor provides a line conditioning and voltage protection circuit for said control and transfer system.

11. The apparatus as set forth in claim 1, wherein said interface is a software process or a hardware interface.

12. The apparatus as set forth in claim 1, wherein said calculation and storage device comprises a facial recognition algorithm for identifying features of said human or said animal, an algorithm for determining the position of said features in three dimensions, and an algorithm for determining the rotation and translation of said features.

13. A method of motion compensating magnetic resonance imaging of a human or animal in real time, comprising the steps of:
a) mounting one or more magnetic-resonance compatible cameras on a magnetic resonance coil of a magnetic resonance imaging device;
b) positioning a set of magnetic-resonance compatible markers on said human or animal;
c) acquiring an image of said markers with said cameras;
d) identifying said markers;
e) determining the position of said markers in three dimensions;
f) determining the rotation and translation of said markers; and
g) modifying geometrical prescriptions of said magnetic resonance imaging device based on said determining of said rotation and said translation.

14. The method as set forth in claim 13, further comprising imaging said markers using magnetic resonance imaging;

15. The method as set forth in claim 14, further comprising transforming the positions of said markers from a coordinate system of one or more of said cameras to a coordinate system of said magnetic resonance imaging device.

16. The method as set forth in claim 13, wherein said positioning comprises attaching spheres of equal or varying but known sizes and spacings to a central sphere, and attaching said central sphere to said human or animal.

17. The method as set forth in claim 13, further comprising utilizing a parallel imaging reconstruction algorithm.

18. A method of motion compensating magnetic resonance imaging of a human or animal in real time, comprising the steps of:
a) mounting at least two magnetic-resonance compatible cameras on a magnetic resonance coil of a magnetic imaging device;
b) acquiring an image of the head of said human or animal with said cameras;
c) identifying features of said head using a facial recognition algorithm;
d) determining the position of said features in three dimensions;
e) determining the rotation and translation of said features; and
f) modifying geometrical prescriptions of said magnetic imaging device based on said determining of said rotation and said translation.

19. The method as set forth in claim 18, wherein said identifying comprises use of at least one of Haar classifiers or a CAMSHIFT algorithm.

20. The method as set forth in claim 18, further comprising utilizing a parallel imaging reconstruction algorithm.
